(19) United States
(12) Patent Application Publication
(10) Pub. No.: US 2021/0382039 A1
(43) Pub. Date: Dec. 9, 2021

(54) METHOD FOR ASSESSING THE ABILITY OF A SUBSTANCE OR OF A COMPOSITION TO PREVENT, SLOW OR ELIMINATE THE SIGNS OF AGEING OF THE SKIN OR OF THE LIPS

(71) Applicants: SOCIETE D'EXPLOITATION DE PRODUITS POUR LES INDUSTRIES CHIMIQUES SEPPIC, PARIS CEDEX 07 (FR); BIOTECHMARINE, Quemper Guezennec (FR)

(72) Inventors: Laetitia CATTUZZATO, Castres (FR); Gaëlle VINCENT, Paris Cedex 07 (FR); Rémi LAVILLE, Quemper Guezennec (FR); Sandrine VINAT, Quemper Guezennec (FR); Cécile BIZE, Paris Cedex 07 (FR)

(21) Appl. No.: 17/291,065

(22) PCT Filed: Nov. 4, 2019
(86) PCT No.: PCT/FR2019/052592

§ 371 Date: May 4, 2021

(30) Foreign Application Priority Data
Nov. 6, 2018 (FR) 1860191

(51) Int. Cl. G01N 33/50; A61Q 19/08; A61K 8/41

(52) U.S. Cl. CPC G01N 33/5023; A61Q 19/08; A61K 8/41; G01N 33/5044; G01N 2333/5412; A61K 2800/92; G01N 2500/10; G01N 2333/938; A61K 2800/74

(57) ABSTRACT

A method for assessing the ability of a chemical substance or of a chemical composition to prevent or to slow the appearance of signs of aging of the human skin or of the lips or even to eliminate the signs; the method including: —a step a) of bringing the chemical substance or the chemical composition into contact with fibroblast cells of the “young” human dermis taken from a culture medium at the passage R6; —a step b) of bringing senescent cells of fibroblasts of the human dermis into contact with the cells of “young” fibroblasts from step a); and—a step c) of measuring the senescence input of the cells of “young” fibroblasts and of comparing it with a reference value.

The present invention relates to a process for evaluating the capacity of a chemical substance (S) or of a chemical composition (C) for preventing or slowing down the appearance of the signs of aging of human skin or the lips or for eliminating said signs.

The present invention also relates to a cosmetic composition and a cosmetic formulation.

Human skin constitutes the first visual image offered to others and, consequently, improving its appearance is often a subject of concern for human beings. The skin is the reflection of a state of well-being, often associated with youthfulness, or, conversely, of a state of fatigue and/or aging. Cosmetic product consumers are thus in search of solutions for attenuating and/or preventing the visible manifestations associated with said aging.

Skin aging is a phenomenon which results from two distinct biological processes:
intrinsic or “chronological” aging under the dependence of genetic characteristics and thus specific to each individual
extrinsic aging influenced by environmental factors, for instance repeated and prolonged exposure to natural or artificial ultraviolet rays (or photoaging), atmospheric pollution, cigarette smoke, various oxidizing agents; various psychological, emotional and/or nervous stresses.

Aging is characterized by metabolic, functional, cellular, architectural and tissue impairments leading to visible external effects. For skin aging, it is a matter of the appearance and/or growth of wrinkles and fine lines, a dull complexion, lack of uniformity of this complexion (dyschromia), or else modification of the texture of the skin (for instance a decrease in the thickness of the skin) and in the properties, notably the biomechanical properties (loss of elasticity), of the skin.

Thus, in the course of time, the renewal of the skin, which is an organ that is under constant renewal, slows down, this phenomenon leading to an accumulation of “old” cells and to impairment of the mechanisms of repair and maintenance of said cells.

The skin cells then go into senescence: they undergo stoppage of the cell cycle, arising after a finite number of divisions, associated with numerous changes and specific phenotypic traits [1].

The most notable changes are:
an increase in the size of the cells
an increase in the size of the cell nuclei
an increase in the lysosomal activity of the cells leading to an increase in autophagy
the production and secretion of bioactive molecules, for instance interleukins, growth factors, matrix-degrading enzymes or reactive oxygen species (ROS); said bioactive molecules constitute the Senescence-Associated Secretory Phenotype (SASP) or the Senescence Messaging Secretome (SMS) [2] [3]
modification of the metabolic activity notably with an increase in the synthesis of matrix metalloproteases (MMP-1), a decrease in procollagen-1 synthesis and an increase in the activity of β-galactosidase (characteristic marker of cellular senescence)
the appearance of DNA damage
modification of the cell cycle and slowing of cell proliferation.

This senescence phenomenon is associated with numerous mechanisms including the contagion of “young” cells with the mediators produced by the neighboring senescent cells.

These molecules secreted by these “old” cells (SMS or SASP) are detected by the neighboring “young” cells not exhibiting the cell modifications described above (morphology, metabolism or proliferation, etc.). These neighboring “young” cells, having been in contact with the molecules secreted by the senescent cells (or SMS), will then in turn go into senescence and exhibit the specific morphological and cellular impairments of skin aging. This phenomenon has recently been described and has been termed “contagious aging” or the “bystander effect” [4].

Methods using co-cultures of different skin cells, exposed to ultraviolet radiation, have been described for studying the susceptibility of human melanocytes, keratinocytes and fibroblasts to induce a “bystander effect” or to be sensitive to this effect [5]. In these methods, the various types of cell cultures were exposed to ultraviolet A rays, and various senescence markers, particularly the oxidizing effects and the DNA damage, were evaluated. It emerges from this study that:
senescence is sparingly induced by direct exposure to ultraviolet A rays on melanocytes, but said melanocytes are sensitive to contagious aging or the “bystander effect”;
the keratinocytes are the cells that are the most resistant to contagious aging or the “bystander effect”;
the fibroblasts exhibited intermediate responses both to induction with ultraviolet A rays and to resistance to contagious aging or the “bystander effect”.

However, this method is characterized by a long implementation time, which is sparingly suited to a use for selecting antiaging active agents in a screening approach, which is very common in the research field.

It appears that the prior art does not disclose any in vitro process for selecting a chemical substance or a chemical composition for preventing or slowing down the appearance of the signs of aging of human skin or the lips or for eliminating said signs, which is liable to be usable at moderate throughput in a screening approach, which does not involve animal cells, which is reproducible, sensitive, discriminating and efficient, and which is suited to the cosmetics market.

There is thus a need to develop a process for selecting a chemical substance or a chemical composition for preventing or slowing down the appearance of the signs of aging of human skin or the lips or for eliminating said signs; said process:
not involving the use of animal cells
being performed over a sufficiently short time (72 hours maximum)
not using any model of clinical studies or of biopsies from clinical studies
and consequently being able to be used in a screening approach, directed toward testing and possibly selecting up to 12 products divided among four culture series.

One solution of the present invention is a process for evaluating the capacity of a chemical substance (S) or of a chemical composition (C) for preventing or slowing down the appearance of the signs of aging of human skin or the lips or for eliminating said signs; said process comprising:
a step a) of placing the chemical substance or the chemical composition in contact with “young” human dermal fibroblast cells taken from a culture medium at the R6 passage,
a step b) of placing human senescent dermal fibroblast cells in contact with “young” fibroblast cells obtained from step a), and
a step c) of measuring the entry into senescence of the “young” fibroblast cells and of comparison with a reference value.

The term “substance” means a molecule defined by a formula, and the term “composition” means a mixture of molecules.

The term “placing in contact” means addition in the same medium.

For the purposes of the present invention, the term “human dermal fibroblasts” denotes cells present in connective tissue, resident in the dermis, and which ensure the coherence and suppleness thereof. It is notably understood by those skilled in the art that, in the course of skin aging, the number of fibroblasts has a tendency to decrease, that the ratio between the collagen III and I fibers changes, and that the elements of the extracellular matrix (collagen, elastic fibers, glycosaminoglycans) decrease. The skin becomes thin, less elastic, and folds or wrinkles appear.

For the purposes of the present invention, the term “Ri passage” denotes the number (i) of subcultures undergone by the cells, i.e.:
19 subcultures for the R19 passage
20 subcultures for the R20 passage
5 subcultures for the R5 passage
6 subcultures for the R6 passage.

For the purposes of the present invention, the term “capacity for preventing or slowing down the appearance of the signs of aging of human skin or the lips or for eliminating said signs” means the capacity for prolonging the duration after which said signs of aging appear on human skin or on the lips.

For the purposes of the invention, the term “signs of aging of human skin or the lips” means any changes in the external appearance of the skin or the lips due to aging, for example wrinkles and fine lines, impairment of the microrelief, lack of elasticity and/or tonicity of the skin, lack of density and/or firmness of human skin or the lips, but also any internal changes in the skin that are not systematically reflected by a modified external appearance, for example any internal degradation of the skin consecutive to exposure to ultraviolet rays.

Depending on the case, the process according to the invention may have one or more of the features below:
the senescent cells are human dermal fibroblast cells taken from a culture medium at the R20 passage,
the senescent cells are human dermal fibroblast cells which have been taken from a culture medium at the R19 passage before undergoing amplification,
the “young” human dermal fibroblast cells taken from a culture medium at the R6 passage are obtained from an amplification of “young” human dermal fibroblast cells taken from a culture medium at the R5 passage,
step c) of measuring the entry into senescence of the “young” fibroblast cells comprises the use of at least two biological markers: at least one biological marker (M1) for SASP/SMS, and at least one biological marker (M2) for senescence,
the biological marker (M1) for SASP/SMS is chosen from the elements of the group consisting of proinflammatory cytokines, and more particularly extracellular interleukin-1 (IL-1), extracellular interleukin-6 (IL-6), extracellular interleukin-8 (IL-8), growth factors, matrix degradation enzymes such as MMP-1 and MMP-3, reactive oxygen species or ROS,
the biological marker (M2) for senescence is chosen from the elements of the group consisting of β-galactosidase, persistent DNA damage, and apoptosis resistance,
the biological marker (M1) is interleukin-6 and the biological marker (M2) is the β-galactosidase enzyme,
in step c), the levels of expression of the two biological markers (M1) and (M2) are compared with at least one reference level of expression for each of these two biological markers,
the signs of aging of human skin or the lips are chosen from the appearance of wrinkles, fine lines or impairment of the microrelief; and/or the appearance of lack of elasticity and/or tonicity; and/or the appearance of lack of density and/or firmness.

For the purposes of the present patent application, the term “biological marker” means a feature which is objectively measured and evaluated as an indicator of normal biological processes, of pathogenic processes or of pharmacological responses to an external intervention. A biological marker may be, for example, a substance whose detection indicates a particular pathological state or, on the contrary, a substance whose detection indicates a particular physiological state.

For the purposes of the present invention, the term “biological marker (M1) for SASP/SMS” denotes a biological marker as defined previously, the variation of expression of which is correlated with the phenomenon of contagious aging or the bystander effect, in the sense of belonging to the group of active molecules contained in the SASP or “senescent associated secreted phenotype”, also known as the SMS or “senescence messaging secretome”.

Among the biological markers (M1) for SASP/SMS for which the level of expression is measured in step e) of the process that is the subject of the present invention, mention may be made of the elements of the group of biological markers (M1) of the group consisting of extracellular interleukin-1 (IL-1), extracellular interleukin-6 (IL-6), interleukin-8 (IL-8), growth factors, matrix degradation enzymes such as MMP-1 and MMP-3, reactive oxygen species or ROS, and most particularly extracellular interleukin-6 (IL-6).

For the purposes of the present patent application, the biological marker IL-6 comprises the human IL-6 gene (NCBI reference: Gene ID: GenBank: JQ250825.1), and also the products of this gene. In a particular embodiment, the biological marker IL-6 consists of one of the products of the human IL-6 gene. The products of the human IL-6 gene comprise the transcript of the human IL-6 gene and the human IL-6 protein. For the purposes of the present patent application, the “transcript of the human IL-6 gene” is the polynucleotide whose sequence has the NCBI reference: GenBank: M54894.1. For the purposes of the present patent application, the term “human IL-6 protein” means the protein whose peptide sequence is the sequence having the NCBI reference: GenBank: AAD13886.1.

For the purposes of the present patent application, the term “biological marker (M2) for senescence” means a biological marker as defined previously, the variation in expression of which is correlated with cellular senescence.

Among the biological markers (M2) for senescence, for which the level of expression is measured in step c) of the process that is the subject of the present invention, mention may be made of the elements of the group of biological markers (M2) consisting of the β-galactosidase enzyme, persistent DNA damage (associated with the detection of γH2AX foci), apoptosis resistance (associated with the detection of p53, p16, p21 and IGFBP5), most particularly the β-galactosidase enzyme.

According to a particular aspect of the process as defined previously, in step c), the biological marker (M1) is interleukin-6 and the biological marker (M2) is the β-galactosidase enzyme.

For each of the biological markers (M1) and (M2), the expression “measurement of the level of expression” generally refers either to a measurement of the amount of gene transcripts or to a measurement of the amount of biological molecules, and more particularly of proteins and metabolites, produced by the human body, or to a level of enzymatic activity.

When the level of expression of the biological marker is measured at the nucleotide level, namely by measuring the amount of products of the gene in its nucleotide form, any method usually used by a person skilled in the art for measuring the amounts of nucleotides may be used, and mention may thus be made of qRTPCR (real-time quantitative polymerase chain reaction), DNA chips and in-situ hybridization.

When the level of expression of the biological marker is measured at the protein, functional or metabolic level, namely by measuring the amount thereof if it is a protein or a metabolite, or by measuring its biological function, any method usually used by a person skilled in the art for measuring amounts of proteins or of metabolites or functionalities may be used and mention may thus be made of ELISA assay, Western blotting, mass spectrometry, immunofluorescence, chromatographic techniques and enzymatic activity.

In the process that is the subject of the present invention, measurement of the expression of the biological marker (M1) for inflammation is more particularly performed at the protein level.

According to a more particular aspect of the process that is the subject of the present invention, when the biological marker (M1) is interleukin-6, measurement of the interleukin-6 expression is performed at the protein level and even more particularly with a measurement using a colorimetric ELISA (enzyme-linked immunosorbent assay) method. This method uses a specific antibody for human IL-6 attached to the bottom of the wells of a measuring plate. The samples to be assayed are added to the wells and the IL-6 present in the samples binds to the immobilized antibody. The wells are then washed and another biotinylated anti-human IL-6 antibody is added.

The wells are once again rinsed to remove the nonbound antibody, and streptavidin conjugated to the enzyme HRP (horseradish peroxidase) is added to the wells. The wells are once again rinsed and the HRP substrate tetramethylbenzidine is added to the wells.

The intensity of the color develops proportionately to the amount of IL-6 bound. The “stop” solution changes the color from blue to yellow, and the color intensity is measured at 450 nm by spectrophotometry. The IL-6 concentration is calculated by referring to a calibration range.

Measurement of the activity of the β-galactosidase enzyme is performed at the metabolic level, and even more particularly with a measurement involving colorimetric enzymatic detection.

Briefly, the β-galactosidase present in the cells reacts specifically with one of its substrates, X-gal (or 5-bromo-4-chloro-3-indolyl-β-D-galactopyranoside (C14H15BrClNO6)) which is deposited on the cell lawns. This substrate is a galactoside, a galactose heteroside, bonded to a substituted indole nucleus. This colorless compound is hydrolyzed by (3-galactosidase, which releases the indole part which then forms by oxidation a water-insoluble blue compound which precipitates at the reaction site. The blue coloration is detected/observed with a photography-capable microscope. For each photograph, the number of marked cells (blue cells) and the total number of visible cells are counted and the number of blue cells is taken as a proportion of the total number of cells for the normalization.

According to and even more particular aspect of the process that is the subject of the present invention, when the biological marker (M1) is interleukin-6, measurement of the interleukin-6 expression is performed at the protein level, and even more particularly with a measurement using a colorimetric ELISA method, and when the biological marker (M2) is β-galactosidase, measurement of the β-galactosidase activity is performed at the metabolic level, and even more particularly with a measurement using colorimetric enzymatic detection.

The term “reference level of expression”” of a biological marker denotes any level of expression of said biological marker used as reference.

In the context of the present invention, a reference level of expression of the biological marker (M1) for SASP/SMS may be obtained by measuring the level of expression of said biological marker (M1):
in the cell culture medium of the “young” human dermal fibroblast cells at the R6 passage (“young” cells at the R6 passage not placed in contact with senescent cells and not treated with the substance or the chemical composition). This measurement of the level of expression of the marker (M1) on the untreated and unstressed cells is noted N1, and/or
in a cell culture medium obtained by placing “young” human dermal fibroblast cells taken at the R6 passage in contact with senescent human dermal fibroblast cells taken at the R20 passage; this measurement of the level of expression of the marker (M1) on the untreated but stressed cells is noted N1₀; and/or
in the cell culture medium obtained on conclusion of the implementation of step b) of the process that is the subject of the present invention, when the substance (S) used in step a) of the process that is the subject of the present invention is a substance chosen from the elements of the group consisting of vitamin E and vitamin C. This measurement of the level of expression of the biological marker (M1) for a reference substance (S) is noted N1Ref.

In the context of the present invention, a reference level of expression of the biological marker (M2) for senescence may be obtained by measuring the level of expression of said biological marker (M2):
in the cell culture medium of the “young” human dermal fibroblast cells at the R6 passage (“young” cells at the R6 passage not placed in contact with senescent cells and not treated with the substance or the chemical composition); this measurement of the level of expression of the biological marker (M2) on the untreated and unstressed cells is noted N2, and/or
in a cell culture medium obtained by placing “young” human dermal fibroblast cells taken at the R6 passage in contact with senescent human dermal fibroblast cells taken at the R20 passage; this measurement of the level of expression of the marker (M2) on the untreated but stressed cells is noted N20; and/or
in the cell culture medium obtained on conclusion of the implementation of step b) of the process that is the subject of the present invention, when the substance (S) used in step a) of the process that is the subject of the present invention is a substance chosen from the elements of the group consisting of vitamin E and vitamin C. This measurement of the level of expression of the biological marker (M2) for a reference substance (S) is noted N2Ref.

By means of the process as defined previously, it is possible to select said substance (S) or said composition (C) as ingredient intended for preventing or slowing down the appearance of the signs of aging of human skin or the lips or for eliminating said signs, and which may be used as such, or for preparing compositions for topical use intended for the cosmetics or dermocosmetic or pharmaceutical industries.

By means of the process as defined previously, a substance (S) or a composition (C) will be selected to be used as such, or for preparing compositions for topical use comprising same, for preventing or slowing down the appearance of the signs of aging of human skin or the lips or for eliminating said signs, if:
the level of expression measured for said substance (S) or for said composition (C), noted N1i, is lower than the reference level of expression of the biological marker (M1) for SASP/SMS N10, and if
the level of expression measured for said substance (S) or for said composition (C), noted N2i, is lower than the reference level of expression of the biological marker (M2) N20 for senescence.

A substance (S) or a composition (C) will be more particularly selected to be used as such, or for preparing cosmetic compositions for topical use comprising same, for preventing or slowing down the appearance of the signs of aging of human skin or the lips or for eliminating said signs, if:
the ratio R1=[(N10−N1i)×100]/[(N10−N1)] is greater than or equal to n1; and if
the ratio R2=[(N20−N2i)×100]/[(N20−N2)] is greater than or equal to n2,
   with n1 greater than or equal to 15, and more particularly greater than or equal to 30, n2 greater than or equal to 60 and more particularly greater than or equal to 70, N1, N10, N2 and N20 being as defined previously, or
   n1 as defined above is more particularly greater than or equal to 15, most particularly greater than or equal to 30, and even more particularly greater than or equal to 40; and n2 as defined above is more particularly greater than or equal to 60, most particularly greater than or equal to 70 and even more particularly greater than or equal to 80.

By means of the process as defined previously, a substance (S) or a composition (C) will be more particularly selected if n1 is greater than or equal to 30 and if n2 is greater than or equal to 70.

A subject of the present invention is also a cosmetic composition (CA) comprising, per 100% of its mass, from 0.1% to 25% by mass of at least one compound of general formula (I):

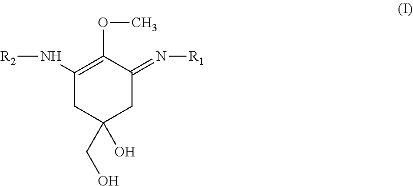

in which:
R₁ represents the radical of formula (Ia)

—CH₂—CO₂H (Ia),

and R₂ represents a hydrogen atom, or
R₁ represents the radical of formula (Ib)

—CH(CO₂H)—CH₂—CH₂—OH (Ib),

and R₂ represents a hydrogen atom, or
R₁ represents the radical of formula (Ia) and R₂ represents the hydroxyethyl radical (Ic):

HO—CH₂—CH₂— (Ic), or
R₁ represents the radical of formula (Ia) and R₂ represents the hydroxymethylethyl radical (Id):

HO—C(CH₃)(CH₂—CH₃) (Id), or
R₁ represents the radical of formula (Ia) and R₂ represents the hydroxypropyl radical (Ie):

HO—CH₂—CH₂—CH₂— (Ie), or
R₁ represents the radical of formula (Ia) and R₂ represents the (E)-propenyl radical (If), or
R₁ represents the radical of formula (Ia) and R₂ represents the (Z)-propenyl radical (Ig), or
R₁ and R₂ both represent the radical of formula (Ia), or
R₁ represents the radical of formula (Ii)

—CH(CO₂H)—CH(OH)—CH₃ (Ii)

and R₂ represents a hydrogen atom, or
R₁ represents the radical of formula (Ii) and R₂ represents a methyl radical, or
R₁ represents the radical of formula (Ib) and R₂ represents a methyl radical, or
R₁ represents the radical of formula (Ia) and R₂ represents the radical of formula (Ij)

—CH(CO₂H)—CH₃ (Ij), or
R₁ represents the radical of formula (Ia) and R₂ represents the radical of formula (Ib), or
R₁ represents the radical of formula (Ia) and R₂ represents the radical of formula (Ik)

—CH(CO₂H)—CH(CH₃)₂ (Ik), or
R₁ represents the radical of formula (Ia) and R₂ represents the radical of formula (Ii), or
R₁ represents the radical of formula (Ia) and R₂ represents the radical of formula (Il)

—CH(CO₂H)—CH₂(CO₂H) (Il), or
R₁ represents the radical of formula (Ia) and R₂ represents the radical of formula (Im)

—CH₂—CH═CH—CO₂H (Im).

According to a particular aspect, a subject of the invention is a cosmetic composition (CA) comprising, per 100% of its mass, from 0.1% to 25% by mass of at least one compound of general formula (I) in which:
R₁ and R₂ both represent the radical of formula (Ia), or
R₁ represents the radical of formula (Ia) and R₂ represents a hydrogen atom, or
R₁ represents the radical of formula (Ia) and R₂ represents the radical (Ic), or
R₁ represents the radical of formula (Ia) and R₂ represents the radical of formula (Ib), or
R₁ represents the radical of formula (Ia) and R₂ represents the radical (If).

According to another particular aspect, a subject of the invention is a composition as defined previously, for preventing or slowing down the appearance of the signs of aging of human skin or the lips or for eliminating said signs.

The cosmetic composition (CA) as defined above may be prepared by mixing the components thereof in the desired mass proportions, at room temperature or above the melting point of the ingredients if necessary, and with moderate mechanical stirring (50 to 150 rpm).

The cosmetic composition (CA) as defined above may also be prepared by any extraction technique known to those skilled in the art, and applied to red algae, and more particularly to algae of the division of Rhodophyta, of the class of Florideophycae, of the order of Acrochaetiales, Acrosymphytales, Ahnfeltiales, Balbianiales, Balliales, Batrachospermales, Bonnemaisoniales, Ceramiales, Colaconematales, Corallinales, Entwisleiales, Florideophyceae incertae sedis, Gelidiales, Gigartinales, Gracilariales, Halymeniales, Hildenbrandiales, Nemaliales, Nemastomatales, Palmariales, Peyssonneliales, Pihiellales, Plocamiales, Rhodachlyales, Rhodogorgonales, Rhodymeniales, Sebdeniales, Sporolithales and Thoreales.

According to a more particular aspect, the cosmetic composition (CA) as defined above may also be prepared via any extraction technique known to those skilled in the art, and applied to red algae of the order of Bonnemaisoniales, of the family of Bonnemaisoniaceae, of the genera/species Asparagopsis armata and Asparagopsis taxiformis.

A final subject of the present invention is a cosmetic formulation for topical use comprising at least one cosmetically acceptable excipient and an effective amount of the cosmetic composition (CA) as defined previously.

According to a particular aspect, the cosmetic formulation according to the invention will be a formulation for preventing or slowing down the appearance of the signs of aging of human skin or the lips or for eliminating said signs.

Said cosmetic formulation for topical use will generally be spread over the surface of the skin to be treated and the skin is then massaged for a few moments.

The expression “for topical use” used in the definition of the cosmetic formulation means that said formulation is used by application to the skin, whether it is a direct application in the case of a cosmetic formulation, or an indirect application when the cosmetic formulation according to the invention is impregnated onto a support intended to be brought into contact with the skin (paper, wipe, textile, transdermal device, etc.).

The term “cosmetically acceptable” used in the definition of the cosmetic formulation for topical use, used in the process which is a subject of the present invention, means, according to the Council of the European Economic Community Directive No. 76/768/EEC of Jul. 27, 1976, amended by Directive No. 93/35/EEC of Jun. 14, 1993, that said formulation for topical use comprises any substance or preparation intended to be brought into contact with the various parts of the human body (epidermis, bodily hair and head hair system, nails, lips and genitals) or with the teeth and oral mucosae, for the purpose, exclusively and mainly, of cleansing them, fragrancing them, modifying the appearance thereof and/or correcting bodily odors thereof and/or protecting them or keeping them in good condition.

The term “effective amount” of composition (A) as defined previously and present in the cosmetic formulation for topical use that is a subject of the present invention means, per 100% of the mass of said cosmetic formulation for topical use, the amount of between 0.1% and 5% by mass, more particularly between 0.1% and 3% by mass, and even more particularly between 0.5% and 2.5% by mass of composition (A).

The cosmetic formulations for topical use according to the invention are generally in the form of aqueous or aqueous-alcoholic or water-glycol solutions, in the form of a suspension, an emulsion, a microemulsion or a nanoemulsion, whether they are of water-in-oil, oil-in-water, water-in-oil-in-water or oil-in-water-in-oil type, or in the form of a powder.

The cosmetic formulations for topical use according to the invention may be packaged in a bottle, in a device of “pump-action bottle” type, in pressurized form in an aerosol device, in a device equipped with a perforated wall such as a grate, or in a device equipped with a ball applicator (known as a “roll-on”).

In general, the composition (CA), present in the cosmetic formulations for topical use according to the invention, is combined with chemical additives normally used in the field of formulations for topical use, such as foaming and/or detergent surfactants, thickening and/or gelling surfactants, thickeners and/or gelling agents, stabilizers, film-forming compounds, solvents and cosolvents, hydrotropic agents, spring or mineral waters, plasticizers, emulsifiers and coemulsifiers, opacifiers, nacreous agents, superfatting agents, sequestrants, chelating agents, oils, waxes, antioxidants, fragrances, essential oils, preserving agents, conditioning agents, deodorants, whitening agents intended for bleaching bodily hairs and the skin, active principles intended to provide a treating and/or protective action with respect to the skin or the hair, sunscreens, mineral fillers or pigments, particles which provide a visual effect or which are intended for encapsulating active agents, exfoliating particles, texturing agents, optical brighteners or insect repellents.

As examples of foaming and/or detergent surfactants that may be combined with composition (CA) in the cosmetic formulations for topical use according to the invention, mention may be made of anionic, cationic, amphoteric or nonionic foaming and/or detergent surfactants.

Among the foaming and/or detergent anionic surfactants, mention may be made of alkali metal salts, alkaline-earth metal salts, ammonium salts, amine salts or amino alcohol salts of alkyl ether sulfates, of alkyl sulfates, of alkylamido ether sulfates, of alkylarylpolyether sulfates, of monoglyceride sulfates, of α-olefin sulfonates, of paraffin sulfonates, of alkyl phosphates, of alkyl ether phosphates, of alkyl sulfonates, of alkylamide sulfonates, of alkylaryl sulfonates, of alkyl carboxylates, of alkylsulfosuccinates, of alkyl ether sulfosuccinates, of alkylamide sulfosuccinates, of alkyl sulfoacetates, of alkyl sarcosinates, of acylisethionates, of N-acyl taurates, of acyl lactylates, of N-acylamino acid derivatives, of N-acyl peptide derivatives, of N-acyl protein derivatives, of N-acyl fatty acid derivatives.

Among the foaming and/or detergent amphoteric surfactants, mention may be made of alkylbetaines, alkylamidobetaines, sultaines, alkylamidoalkylsulfobetaines, imidazoline derivatives, phosphobetaines, amphopolyacetates and amphopropionates.

Among the foaming and/or detergent cationic surfactants, mention may be made particularly of quaternary ammonium derivatives.

Among the foaming and/or detergent nonionic surfactants, mention may be made more particularly of alkylpolyglycosides including a linear or branched, saturated or unsaturated aliphatic radical, including from 8 to 16 carbon atoms, for instance octyl polyglucoside, decyl polyglucoside, undecylenyl polyglucoside, dodecyl polyglucoside, tetradecyl polyglucoside, hexadecyl polyglucoside, 1,12-dodecanediyl polyglucoside; ethoxylated hydrogenated castor oil derivatives, for instance the product sold under the INCI name PEG-40 hydrogenated castor oil; polysorbates, for instance Polysorbate 20, Polysorbate 40, Polysorbate 60, Polysorbate 70, Polysorbate 80 and Polysorbate 85; coconut kernel amides; N-alkylamines.

As examples of thickening and/or gelling surfactants that may be combined with composition (CA) in the cosmetic formulations for topical use according to the invention, mention may be made of optionally alkoxylated alkylpolyglycoside fatty esters, for instance ethoxylated methylpolyglucoside esters, such as the PEG 120 methyl glucose trioleate and the PEG 120 methyl glucose dioleate sold, respectively, under the names Glutamate™ LT and Glutamate™ DOE120; alkoxylated fatty esters, such as the PEG 150 pentaerythrityl tetrastearate sold under the name Crothix™ DS53, the PEG 55 propylene glycol oleate sold under the name Antil™ 141; fatty-chain polyalkylene glycol carbamates, for instance the PPG-14 laureth isophoryl dicarbamate sold under the name Elfacos™ 1211, the PPG-14 palmeth-60 hexyl dicarbamate sold under the name Elfacos™ GT2125.

As examples of thickeners and/or gelling agents that may be combined with composition (CA) in the cosmetic formulations for topical use according to the invention, mention may be made of linear or branched or crosslinked polymers of polyelectrolyte type, such as the partially or totally salified acrylic acid homopolymer, the partially or totally salified methacrylic acid homopolymer, the partially or totally salified 2-methyl[(1-oxo-2-propenyl)amino]-1-propanesulfonic acid (AMPS) homopolymer, copolymers of acrylic acid and of AMPS, copolymers of acrylamide and of AMPS, copolymers of vinylpyrrolidone and of AMPS, copolymers of AMPS and of (2-hydroxyethyl) acrylate, copolymers of AMPS and of (2-hydroxyethyl) methacrylate, copolymers of AMPS and of hydroxyethylacrylamide, copolymers of AMPS and of N,N-dimethylacrylamide, copolymers of AMPS and of tris(hydroxymethyl)acrylamidomethane (THAM), copolymers of acrylic or methacrylic acid and of (2-hydroxyethyl) acrylate, copolymers of acrylic or methacrylic acid and of (2-hydroxyethyl) methacrylate, copolymers of acrylic or methacrylic acid and of hydroxyethylacrylamide, copolymers of acrylic or methacrylic acid and of THAM, copolymers of acrylic or methacrylic acid and of N,N-dimethylacrylamide, terpolymers of acrylic or methacrylic acid, of AMPS and of (2-hydroxyethyl) acrylate, terpolymers of acrylic or methacrylic acid, of AMPS and of (2-hydroxyethyl) methacrylate, terpolymers of acrylic or methacrylic acid, of AMPS and of THAM, terpolymers of acrylic or methacrylic acid, of AMPS and of N,N-dimethylacrylamide, terpolymers of acrylic or methacrylic acid, of AMPS and of acrylamide, copolymers of acrylic acid or methacrylic acid and of alkyl acrylates, the carbon chain of which comprises between 4 and 30 carbon atoms and more particularly between 10 and 30 carbon atoms, copolymers of AMPS and of alkyl acrylates, the carbon chain of which comprises between 4 and 30 carbon atoms and more particularly between 10 and 30 carbon atoms, linear, branched or crosslinked terpolymers of at least one monomer bearing a free, partially salified or totally salified strong acid function, with at least one neutral monomer, and at least one monomer of formula (VIII):

CH₂═C(R′₃)—C(═O)—[CH₂—CH₂—O]ₙ—R′₄ (VIII)

in which R′3 represents a hydrogen atom or a methyl radical, R′4 represents a linear or branched alkyl radical including from 8 to 30 carbon atoms and n represents a number greater than or equal to 1 and less than or equal to 50.

The linear or branched or crosslinked polymers of polyelectrolyte type that may be combined with composition (CA) in the cosmetic formulations for topical use according to the invention may be in the form of an aqueous solution or suspension, a water-in-oil emulsion, an oil-in-water emulsion or a powder. The linear or branched or crosslinked polymers of polyelectrolyte type that may be combined with the compound of formula (I) or with composition (C1) in the cosmetic formulations for topical use according to the invention may be chosen from the products sold under the names Simulgel™ EG, Simulgel™EPG, Sepigel™ 305, Simulgel™ 600, Simulgel™ NS, Simulgel™ INS 100, Simulgel™ FL, Simulgel™ A, Simulgel™ SMS 88, Sepinov™ EMT 10, Sepiplus™ 400, Sepiplus™ 265, Sepiplus™ S, Sepimax™ Zen, Aristoflex™ AVC, Aristoflex™ AVS, Novemer™ EC-1, Novemer™ EC 2, Aristoflex™ HMB, Cosmedia™ SP, Flocare™ ET 25, Flocare™ ET 75, Flocare™ ET 26, Flocare™ ET 30, Flocare™ ET 58, Flocare™ PSD 30, Viscolam™ AT 64 and Viscolam™ AT 100.

As examples of thickening and/or gelling agents at that may be combined with composition (CA) in the cosmetic formulations for topical use according to the invention, mention may be made of polysaccharides consisting only of monosaccharides, such as glucans or glucose homopolymers, glucomannoglucans, xyloglycans, galactomannans of which the degree of substitution (DS) of the D-galactose units on the main D-mannose chain is between 0 and 1, and more particularly between 1 and 0.25, such as galactomannans originating from cassia gum (DS=⅕), locust bean gum (DS=¼), tara gum (DS=⅓), guar gum (DS=½) or fenugreek gum (DS=1).

As examples of thickening and/or gelling agents that may be combined with composition (CA) in the cosmetic formulations for topical use according to the invention, mention may be made of polysaccharides consisting of monosaccharide derivatives, such as sulfated galactans and more particularly carrageenans and agar, uronans and more particularly algins, alginates and pectins, heteropolymers of monosaccharides and uronic acids, and more particularly xanthan gum, gellan gum, gum arabic exudates and karaya gum exudates, or glucosaminoglycans.

As examples of thickening and/or gelling agents that may be combined with composition (CA) in the cosmetic formulations for topical use according to the invention, mention may be made of cellulose, cellulose derivatives such as methyl cellulose, ethyl cellulose or hydroxypropyl cellulose, silicates, starch, hydrophilic starch derivatives, or polyurethanes.

As examples of stabilizers that may be combined with composition (CA) in the cosmetic formulations for topical use according to the invention, mention may be made of microcrystalline waxes, and more particularly ozokerite, mineral salts such as sodium chloride or magnesium chloride, silicone polymers such as polysiloxane polyalkyl polyether copolymers.

As examples of solvents that may be combined with composition (CA) in the cosmetic formulations for topical use according to the invention, mention may be made of water, organic solvents, such as glycerol, diglycerol, glycerol oligomers, ethylene glycol, propylene glycol, butylene glycol, 1,3-propanediol, 1,2-propanediol, hexylene glycol, diethylene glycol, xylitol, erythritol, sorbitol, water-soluble alcohols, such as ethanol, isopropanol or butanol, or mixtures of water and of said organic solvents.

As examples of thermal or mineral waters that may be combined with composition (CA) in the cosmetic formulations for topical use according to the invention, mention may be made of thermal or mineral waters having a mineralization of at least 300 mg/l, in particular Avene water, Vittel water, Vichy basin water, Uriage water, La Roche-Posay water, La Bourboule water, Enghien-les-Bains water, Saint-Gervais-les-Bains water, Néris-les-Bains water, Allevard-les-Bains water, Digne water, Maizières water, Neyrac-les-Bains water, Lons-le-Saunier water, Rochefort water, Saint Christau water, Les Fumades water and Tercis-les-Bains water.

As examples of hydrotropic agents that may be combined with composition (CA) in the cosmetic formulations for topical use according to the invention, mention may be made of xylenesulfonates, cumenesulfonates, hexyl polyglucoside, 2-ethylhexyl polyglucoside or n-heptyl polyglucoside.

As examples of emulsifying surfactants that may be combined with composition (CA) in the cosmetic formulations for topical use according to the invention, mention may be made of nonionic surfactants, anionic surfactants or cationic surfactants.

As examples of emulsifying nonionic surfactants that may be combined with composition (CA) in the cosmetic formulations for topical use according to the invention, mention may be made of fatty acid esters of sorbitol, for instance the products sold under the names Montane™ 40, Montane™ 60, Montane™ 70, Montane™ 80 and Montane™ 85; compositions comprising glyceryl stearate and stearic acid ethoxylated with between 5 mol and 150 mol of ethylene oxide, for instance the composition comprising stearic acid ethoxylated with 135 mol of ethylene oxide and glyceryl stearate sold under the name Simulsol™ 165; mannitan esters, ethoxylated mannitan esters; sucrose esters; methyl glucoside esters; alkyl polyglycosides including a linear or branched, saturated or unsaturated aliphatic radical, and including from 14 to 36 carbon atoms, for instance tetradecyl polyglucoside, hexyldecyl polyglucoside, octadecyl polyglucoside, hexyldecyl polyxyloside, octadecyl polyxyloside, eicosyl polyglucoside, dodecosyl polyglucoside, 2-octyldodecyl polyxyloside, 12-hydroxystearyl polyglucoside; compositions of linear or branched, saturated or unsaturated fatty alcohols including from 14 to 36 carbon atoms and of alkyl polyglycosides as described previously, for example the compositions sold under the brand names Montanov™ 68, Montanov™ 14, Montanov™ 82, Montanov™ 202, Montanov™ S, Montanov™ WO18, Montanov™ L, Fluidanov™ 20× and Easynov™.

As examples of anionic surfactants that may be combined with composition (CA) in the cosmetic formulations for topical use according to the invention, mention may be made of glyceryl stearate citrate, cetearyl sulfate, soaps such as sodium stearate or triethanolammonium stearate, or N-acyl derivatives of salified amino acids, for instance stearoyl glutamate.

As examples of emulsifying cationic surfactants that may be combined with composition (CA) in the cosmetic formulations for topical use according to the invention, mention may be made of amine oxides, quaternium-82 and the surfactants described in patent application WO 96/00719 and mainly those whose fatty chain comprises at least 16 carbon atoms.

As examples of opacifiers and/or nacreous agents that may be combined with composition (CA) in the cosmetic formulations for topical use according to the invention, mention may be made of sodium palmitate, sodium stearate, sodium hydroxystearate, magnesium palmitate, magnesium stearate, magnesium hydroxystearate, ethylene glycol monostearate, ethylene glycol distearate, polyethylene glycol monostearate, polyethylene glycol distearate and fatty alcohols including from 12 to 22 carbon atoms.

As examples of texturing agents that may be combined with composition (CA) in the cosmetic formulations for topical use according to the invention, mention may be made of N-acylamino acid derivatives, for instance lauroyl lysine sold under the name Aminohope™ LL, octenyl starch succinate sold under the name Dryflo™, myristyl polyglucoside sold under the name Montanov™ 14, cellulose fibers, cotton fibers, chitosan fibers, talc, sericite and mica.

As examples of deodorants that may be combined with composition (CA) in the cosmetic formulations for topical use according to the invention, mention may be made of alkaline silicates, zinc salts such as zinc sulfate, zinc gluconate, zinc chloride or zinc lactate; quaternary ammonium salts such as cetyltrimethylammonium salts or cetylpyridinium salts; glycerol derivatives such as glyceryl caprate, glyceryl caprylate and polyglyceryl caprate; 1,2-decanediol, 1,3-propanediol; salicylic acid; sodium bicarbonate; cyclodextrins; metallic zeolites; Triclosan™; aluminum bromohydrate, aluminum chlorohydrates, aluminum chloride, aluminum sulfate, aluminum zirconium chlorohydrates, aluminum zirconium trichlorohydrate, aluminum zirconium tetrachlorohydrate, aluminum zirconium pentachlorohydrate, aluminum zirconium octachlorohydrate, aluminum sulfate, sodium aluminum lactate, complexes of aluminum chlorohydrate and of glycol, such as the complex of aluminum chlorohydrate and of propylene glycol, the complex of aluminum dichlorohydrate and of propylene glycol, the complex of aluminum sesquichlorohydrate and of propylene glycol, the complex of aluminum chlorohydrate and of polyethylene glycol, the complex of aluminum dichlorohydrate and of polyethylene glycol, or the complex of aluminum sesquichlorohydrate and of polyethylene glycol.

As examples of oils that may be combined with composition (CA) in the cosmetic formulations for topical use according to the invention, mention may be made of mineral oils such as liquid paraffin, liquid petroleum jelly, isoparaffins or white mineral oils; oils of animal origin such as squalene or squalane; plant oils, such as phytosqualane, sweet almond oil, coconut kernel oil, castor oil, jojoba oil, olive oil, rapeseed oil, groundnut oil, sunflower oil, wheat germ oil, corn germ oil, soybean oil, cotton oil, alfalfa oil, poppy oil, pumpkin oil, evening primrose oil, millet oil, barley oil, rye oil, safflower oil, candlenut oil, passionflower oil, hazelnut oil, palm oil, shea butter, apricot kernel oil, beauty-leaf oil, sisymbrium oil, avocado oil, calendula oil, oils derived from flowers or vegetables, ethoxylated plant oils; synthetic oils, for instance fatty acid esters such as butyl myristate, propyl myristate, isopropyl myristate, cetyl myristate, isopropyl palmitate, octyl palmitate, butyl stearate, hexadecyl stearate, isopropyl stearate, octyl stearate, isocetyl stearate, dodecyl oleate, hexyl laurate, propylene glycol dicaprylate, esters derived from lanolic acid, such as isopropyl lanolate, isocetyl lanolate, fatty acid monoglycerides, diglycerides and triglycerides, for instance glyceryl triheptanoate, alkylbenzoates, hydrogenated oils, poly(α-olefins), polyolefins such as poly(isobutane), synthetic isoalkanes, for instance isohexadecane, isododecane, perfluorinated oils; silicone oils, for instance dimethylpolysiloxanes, methylphenylpolysiloxanes, silicones modified with amines, silicones modified with fatty acids, silicones modified with alcohols, silicones modified with alcohols and fatty acids, silicones modified with polyether groups, epoxy-modified silicones, silicones modified with fluoro groups, cyclic silicones and silicones modified with alkyl groups. In the present patent application, the term “oils” refers to compounds and/or mixtures of compounds which are water-insoluble, and which have a liquid appearance at a temperature of 25° C.

As examples of waxes that may be combined with composition (CA) in the cosmetic formulations for topical use according to the invention, mention may be made of beeswax, carnauba wax, candelilla wax, ouricury wax, Japan wax, cork fiber wax, sugarcane wax, paraffin waxes, lignite waxes, microcrystalline waxes, lanolin wax; ozokerite; polyethylene wax; silicone waxes; plant waxes; fatty alcohols and fatty acids that are solid at room temperature; glycerides that are solid at room temperature. In the present patent application, the term “waxes” refers to compounds and/or mixtures of compounds which are water-insoluble, and which have a solid appearance at a temperature of greater than or equal to 45° C.

As examples of active principles that may be combined with composition (CA) in the cosmetic formulations for topical use according to the invention, mention may be made of vitamins and derivatives thereof, notably esters thereof, such as retinol (vitamin A) and esters thereof (for example retinyl palmitate), ascorbic acid (vitamin C) and esters thereof, sugar derivatives of ascorbic acid (such as ascorbyl glucoside), tocopherol (vitamin E) and esters thereof (such as tocopheryl acetate), vitamin B3 or B10 (niacinamide and derivatives thereof); compounds showing a lightening or depigmenting action on the skin, such as w-undecylenoyl phenylalanine sold under the name Sepiwhite™ MSH, Sepicalm™ VG, the glyceryl monoester and/or diester of ω-undecylenoyl phenylalanine, ω-undecylenoyl dipeptides, arbutin, kojic acid, hydroquinone; compounds showing a calmative action, notably Sepicalm™ S, allantoin and bisabolol; antiinflammatory agents; compounds showing moisturizing action, such as urea, hydroxyureas, glycerol, polyglycerols, glycerol glucoside, diglycerol glucoside, polyglyceryl glucosides, xylityl glucoside; polyphenol-rich plant extracts such as grape extracts, pine extracts, wine extracts and olive extracts; compounds showing slimming or lipolytic action such as caffeine or derivatives thereof, Adiposlim™, Adipoless™, fucoxanthin; N-acyl proteins; N-acyl peptides such as Matrixyl™; N-acylamino acids; N-acyl partial protein hydrolyzates; amino acids; peptides; total protein hydrolyzates; soybean extracts, for example Raffermine™; wheat extracts, for example Tensine™ or Gliadine™; plant extracts, such as tannin-rich plant extracts, isoflavone-rich plant extracts or terpene-rich plant extracts; freshwater or marine water algal extracts; marine plant extracts; marine extracts in general such as corals; essential waxes; bacterial extracts; ceramides; phospholipids; compounds showing antimicrobial action or purifying action, such as Lipacide™ C8G, Lipacide™ UG, Sepicontrol™ A5; Octopirox™ or Sensiva™ SC50; compounds showing an energizing or stimulating property such as Physiogenyl™, panthenol and derivatives thereof such as Sepicap™ MP; antiaging active agents such as Sepilift™ DPHP, Lipacide™ PVB, Sepivinol™ Sepivital™, Manoliva™, Phyto-Age™, Timecode™; Survicode™; anti-photoaging active agents; agents for protecting the integrity of the dermo-epidermal junction; agents for increasing the synthesis of components of the extracellular matrix, such as collagen, elastins and glycosaminoglycans; active agents acting favorably on chemical cellular communication, such as cytokines, or on physical cellular communication, such as integrins; active agents creating a sensation of “heating” on the skin, such as microcirculation activators (such as nicotinic acid derivatives) or products that create a sensation of “freshness” on the skin (such as menthol and derivatives thereof); active agents which improve the skin microcirculation, for example venotonic agents; draining active agents; decongestant active agents such as Ginkgo biloba, ivy, common horse chestnut, bamboo, Ruscus, butcher's-broom, Centella asiatica, fucus, rosemary or willow extracts; skin tanning or browning agents, for example dihydroxyacetone (DHA), erythrulose, mesotartaric aldehyde, glutaraldehyde, glyceraldehyde, alloxan or ninhydrin, plant extracts, for example extracts of red woods of the genus Pterocarpus and of the genus Baphia, for instance Pteropcarpus santalinus, Pterocarpus osun, Pterocarpus soyauxii, Pterocarpus erinaceus, Pterocarpus indicus or Baphia nitida, such as those described in European patent application EP 0 971 683; agents known for their action in facilitating and/or accelerating tanning and/or browning of human skin, and/or for their action in coloring human skin, for example carotenoids (and more particularly beta-carotene and gamma-carotene), the product sold under the brand name Carrot oil (INCI name: Daucus carrota, Helianthus annuus sunflower oil) by the company Provital, which contain carotenoids, vitamin E and vitamin K; tyrosine and/or derivatives thereof, known for their effect on accelerating the tanning of human skin in combination with exposure to ultraviolet radiation, for example the product sold under the brand name SunTan Accelerator™ by the company Provital, which contains tyrosine and riboflavins (vitamin B), the tyrosine and tyrosinase complex sold under the brand name Zymo Tan Complex by the company Zymo Line, the product sold under the brand name MelanoBronze™ (INCI name: Acetyl tyrosine, monk's pepper extract (Vitex Agnus-castus)) by the company Mibelle, which contains acetyl tyrosine, the product sold under the brand name Unipertan VEG-24/242/2002 (INCI name: Butylene glycol and acetyl tyrosine and hydrolyzed vegetable protein and adenosine triphosphate) by the company Unipex, the product sold under the brand name Try-Excell™ (INCI name: Oleoyl tyrosine and Luffa cylindrica (seed) oil and oleic acid) by the company Sederma, which contains extracts of marrow seeds (or loofah oil), the product sold under the brand name Actibronze™ (INCI name: Hydrolyzed wheat protein and acetyl tyrosine and copper gluconate) by the company Alban Muller, the product sold under the brand name Tyrostan™ (INCI name: Potassium caproyl tyrosine) by the company Synerga, the product sold under the brand name Tyrosinol (INCI name: Sorbitan isostearate, glyceryl oleate, caproyl tyrosine) by the company Synerga, the product sold under the brand name InstaBronze™ (INCI name: Dihydroxyacetone and acetyl tyrosine and copper gluconate) sold by the company Alban Muller, the product sold under the brand name Tyrosilane (INCI name: Methylsilanol and acetyl tyrosine) by the company Exymol; peptides known for their effect on activating melanogenesis, for example the product sold under the brand name Bronzing SF peptide powder (INCI name: Dextran and octapeptide-5) by the company Infinitec Activos, the product sold under the brand name Melitane (INCI name: Glycerin and Aqua and Dextran and Acetyl Hexapeptide-1) comprising acetyl hexapeptide-1 known for its α-MSH agonist action, the product sold under the brand name Melatimes Solutions™ (INCI name: Butylene glycol, Palmitoyl Tripeptide-40) by the company Lipotec, sugars and sugar derivatives, for example the product sold under the brand name Lanositol™ (INCI name: inositol) by the company Provital, the product sold under the brand name Thalitan™ (or Phycosaccharide™ AG) by the company CODIF International (INCI name: Aqua and hydrolyzed algin (Laminaria digitata) and magnesium sulfate and manganese sulfate) containing an oligosaccharide of marine origin (guluronic acid and mannuronic acid chelated with magnesium and manganese ions), the product sold under the brand name Melactiva™ (INCI name: Maltodextrin, Mucuna pruriens seed extract) by the company Alban Muller, flavonoid-rich compounds, for instance the product sold under the brand name Biotanning (INCI name: Hydrolyzed citrus Aurantium dulcin fruit extract) by the company Silab and known to be rich in lemon flavonoids (of the hesperidin type); agents intended for treating head hair and/or bodily hair, for example agents for protecting the melanocytes of the hair follicle, intended to protect said melanocytes against cytotoxic agents responsible for the senescence and/or apoptosis of said melanocytes, such as mimetics of DOPAchrome tautomerase activity, selected from those described in the European patent application published under the number EP 1 515 688 A2, the synthetic SOD mimetic molecules, for example manganese complexes, antioxidant compounds, for example cyclodextrin derivatives, silicon-containing compounds derived from ascorbic acid, lysine or arginine pyrrolidone carboxylate, combinations of mono- and diesters of cinnamic acid and of vitamin C, and more generally those mentioned in the European patent application published under the number EP 1 515 688 A2.

As examples of antioxidants that may be combined with composition (CA) in the cosmetic formulations for topical use according to the invention, mention may be made of EDTA and salts thereof, citric acid, tartaric acid, oxalic acid, BHA (butylhydroxyanisole), BHT (butylhydroxytoluene), tocopherol derivatives such as tocopheryl acetate, mixtures of antioxidant compounds such as Dissolvine [symbol] GL 47S sold by the company AkzoNobel under the INCI name: Tetrasodium Glutamate Diacetate.

As examples of sunscreens that may be combined with composition (CA) in the cosmetic formulations for topical use according to the invention, mention may be made of all those appearing in the Cosmetic Directive 76/768/EEC, amended, Annex VII. Among the organic sunscreens that may be combined with the compound of formula (I) or with composition (C1) in the cosmetic formulations for topical use according to the invention, mention may be made of the family of benzoic acid derivatives, for instance para-aminobenzoic acids (PABA), notably monoglyceryl esters of PABA, ethyl esters of N,N-propoxy PABA, ethyl esters of N,N-diethoxy PABA, ethyl esters of N,N-dimethyl PABA, methyl esters of N,N-dimethyl PABA and butyl esters of N,N-dimethyl PABA; the family of anthranilic acid derivatives, for instance homomenthyl-N-acetyl anthranilate; the family of salicylic acid derivatives, for instance amyl salicylate, homomenthyl salicylate, ethylhexyl salicylate, phenyl salicylate, benzyl salicylate and p-isopropanolphenyl salicylate; the family of cinnamic acid derivatives, for instance ethylhexyl cinnamate, ethyl-4-isopropyl cinnamate, methyl 2,5-diisopropylcinnamate, p-methoxypropyl cinnamate, p-methoxyisopropyl cinnamate, p-methoxyisoamyl cinnamate, p-methoxyoctyl cinnamate (p-methoxy 2-ethylhexyl cinnamate), p-methoxy-2-ethoxyethyl cinnamate, p-methoxycyclohexyl cinnamate, ethyl-α-cyano-β-phenyl cinnamate, 2-ethylhexyl- [symbol] α-cyano-β-phenyl cinnamate or glyceryl di-para-methoxymono-2-ethylhexanoyl cinnamate; the family of benzophenone derivatives, for instance 2,4-dihydroxybenzophenone, 2,2′-dihydroxy-4-methoxybenzophenone, 2,2′,4,4′-tetrahydroxybenzophenone, 2-hydroxy-4-methoxybenzophenone, 2-hydroxy-4-methoxy-4′-methylbenzophenone, 2-hydroxy-4-methoxybenzophenone-5-sulfonate, 4-phenylbenzophenone, 2-ethylhexyl 4′-phenylbenzophenone-2-carboxylate, 2-hydroxy-4-n-octyloxybenzophenone, 4-hydroxy-3-carboxybenzophenone; 3-(4′-methylbenzylidene)-d,l-camphor, 3-(benzylidene)-d,l-camphor, camphor benzalkonium methosulfate; urocanic acid, ethyl urocanate; the family of sulfonic acid derivatives, for instance 2-phenylbenzimidazole-5-sulfonic acid and salts thereof; the family of triazine derivatives, for instance hydroxyphenyltriazine, ethylhexyloxyhydroxyphenyl-4-methoxyphenyltriazine, 2,4,6-trianillino(p-carbo-2′-ethylhexyl-1′-oxy)-1,3,5-triazine, 4,4-((6-(((1,1-dimethylethyl)amino)carbonyl)phenyl)amino)-1,3,5-triazine-2,4-diyldiimino)bis(2-ethylhexyl) benzoate, 2-phenyl-5-methylbenzoxazole, 2,2′-hydroxy-5-methylphenylbenzotriazole, 2-(2′-hydroxy-5′-t-octylphenyl)benzotriazole, 2-(2′-hydroxy-5′-methyphenyl)benzotriazole; dibenzazine; dianisoylmethane, 4-methoxy-4″-t-butylbenzoylmethane; 5-(3,3-dimethyl-2-norbornylidene)-3-pentan-2-one; the family of diphenylacrylate derivatives, for instance 2-ethylhexyl 2-cyano-3,3-diphenyl-2-propenoate, ethyl 2-cyano-3,3-diphenyl-2-propenoate; the family of polysiloxanes, for instance benzylidene siloxane malonate.

Among the mineral sunscreens, also known as “mineral sunblocks”, that may be combined with composition (CA) in the cosmetic formulations for topical use according to the invention, mention may be made of titanium oxides, zinc oxides, cerium oxide, zirconium oxide, yellow, red or black iron oxides, and chromium oxides. These mineral sunblocks may or may not be micronized, may or may not have undergone surface treatments and may optionally be in the form of aqueous or oily predispersions.

The experimental section that follows illustrates the invention without, however, limiting it.

### A-1)—Choice of the Selection Process

### A-1-1) Choice of the Type of Senescence Induction

Four different types of senescence induction were studied:
Acute exposure of “young” fibroblasts taken at the R6 passage to ultraviolet A rays; several radiation doses were tested: 5, 10, 15 or 20 J/cm², for one exposure per day;
Chronic exposure of “young” fibroblasts taken at the R6 passage to ultraviolet A rays; several radiation doses were tested: 3×5 J/cm² or 6×2.5 J/cm², for two exposures per day;
Use of fibroblasts “artificially aged” by successive replications up to the R18 passage;
Use of a pool of fibroblasts obtained from elderly donors (58 years old on average)
   It should be noted that the young fibroblasts (R6 passage) and the senescent fibroblasts (R20 passage) are obtained from the same donors and from the same batch of cells. The fibroblasts taken at the R20 passage were obtained after successive replications of the R6 cells.

To evaluate the most suitable type of induction, the fibroblast cells, whether taken at the R6 passage or at the R20 passage, were seeded in 48-well plates at 15 000 or 10 000 cells/well, in a standard culture medium for fibroblasts, at 37° C. in a humid atmosphere containing 5% CO₂.

Each condition was performed in quadruplicate.

The contagious aging or bystander effect was then evaluated by measuring the amount of extracellular IL-6 and the β-galactosidase activity:
for the chronic or acute UVA exposure
at 96 h and 144 h for the use of the pool of fibroblasts obtained from elderly donors
at 24 h and 48 h for the replication-induced senescence.

The results obtained then made it possible to calculate the degrees of change of the amount of extracellular IL-6 and of the β-galactosidase activity, and to collect qualitative observations; all these results are collated in table 1 below.

TABLE 1

| | | | | Contact with |
|---|---|---|---|---|
| Type of | Acute UVA | Chronic UVA | Replicative | elderly |
| induction | exposure | exposure | senescence | fibroblasts |
| Induction | 5-10-15-20 | 3 × 5 J/cm² or | MCR20 | Young pool vs |
| parameters | J/cm² | 6 × 2.5 J/cm² | | old pool |
| Biological | Rate of IL-6 | Not measurable | Degree of | Degree of |
| marker IL-6 | increase >100% | | change after 48 | change after 96 |
| | from 4 hours | | hours: 59% | hours: 230% |
| Biological | Marking too | Very little | Degree of | Degree of |
| marker | high because of | marking for the | change after 48 | change after 48 |
| β-galactosidase | excessive | negative Control | hours: 378% | hours: observed |
| | confluence | and UVA | | decrease |

Only the replication of the fibroblasts up to the R20 passage made it possible to observe an increase in IL-6 secretion and an increase in β-galactosidase marking characteristic of a phenotype of contagious aging or bystander effect.

The above test thus made it possible to select the successive replication as the type of senescence induction for the model mimicking contagious aging.

### A-1-2) Choice of the Most Suitable Model for Mimicking Contagious Aging

Various methods were used to select the one which was the most suitable for observing the “bystander effect” on the fibroblast cells taken at the R6 and R20 passages:
indirect coculture, i.e. with insertion, of the R6 young fibroblast cells and of the R20 senescent fibroblast cells in standard culture medium for fibroblasts
direct coculture of these cells in the same well with marking of the senescent cells taken at the R20 passage to differentiate them
use of MCR20 media conditioned with R20 senescent fibroblast cells, placed in contact with “young” fibroblasts taken at the R6 passage.

Description of the Protocols Used for Determining the Most Suitable Model for Evaluating the Contagious Aging:

Model of Indirect Coculture Using Inserts:

Normal human fibroblasts taken at the R5 and R19 passages were amplified, respectively, up to the respective R6 and R20 passages and then seeded in 24-well plates at 30 000 cells/well for the R6 and in an insert at 15 000 cells/well for the R20.

After 72 hours of amplification in inserts and in 24-well plates, the inserts, containing the R20 cells, were deposited in the 24-well plates containing the R6 cells and placed in contact for 24 hours and 4 days.

After these incubations of 24 hours and 4 days, the following senescence markers were evaluated:
the β-galactosidase activity by staining
the amount of interleukin-6 by ELISA assay

Model of Direct Coculture:

Normal human fibroblasts taken at the R5 and R19 passages were amplified, respectively, up to the respective R6 and R20 passages and then seeded in Petri dishes at 0.3 M/Petri dish.

After 5 days of amplification in Petri dishes, the cell lawns of the cells at the R20 passage were fluorescence-marked (Alexa Fluor® 488 NHS Ester marker at 5 mM). They were then seeded in 48-well plates with non-senescent and unmarked R6 cells in a 1/1 ratio (i.e. 7500 cells/well for the two passages). The cells thus seeded were then incubated for 48 hours.

After this incubation for 48 hours, the following senescence markers were evaluated:
the β-galactosidase activity by staining
the amount of interleukin-6 by ELISA assay

### Model Using Conditioned Media:

Normal human fibroblasts taken at the R19 passage were amplified up to the R20 passage and then seeded in Petri dishes at 0.3 M/Petri dish.

After amplification for 4 days in Petri dishes, the culture medium of all the dishes was changed and then recovered after 48 hours of incubation. This recovered medium corresponds to the medium conditioned with senescent cells containing the SMS mediators (MCR20).

In parallel, R6 “young” fibroblasts were seeded in 48-well plates at 10 000 and 15 000 cells/well. After 72 hours of incubation, the MCR20 conditioned medium was deposited on these cells. The cells thus treated with the medium conditioned with senescent cells were incubated for 24 hours and 48 hours.

The following senescence markers were then evaluated:
the β-galactosidase activity by staining at 48 hours
the amount of interleukin-6 by ELISA assay at 24 hours

### CONCLUSIONS

Conclusive and reliable results were revealed for the sole model of contagious aging, using the conditioned media: increase in interleukin-6, and modification of their metabolic activity (increase in β-galactosidase activity). These modifications are characteristic of senescence of “young” cells treated with the medium conditioned with aged cells.

### A-2)—Definition of the Optimum Process

The particular process thus comprises the following steps:
Step i) of culturing of culturing human dermal fibroblasts, taken at the R19 passage in T25 flasks,
Step ii) of amplification of the cells obtained on conclusion of step i) to obtain a medium conditioned with senescent cells or “MC R20”.
   Amplification of the cells obtained on conclusion of step i) is performed by seeding said cells in Petri dishes at 0.3 M/Petri dish. After amplification for 4 days in Petri dishes, the culture medium of all the dishes was changed and then recovered after 48 hours of incubation. This recovered medium corresponds to the medium conditioned with senescent cells containing the SMS mediators (MCR20).
Step iii) of culturing human dermal fibroblasts, taken at the R5 passage in T25 flasks,
a step iv) of amplification of the cells obtained on conclusion of step iii) to obtain to obtain “young” or “R6” cells in the amplification phase,
   Steps iii) and iv) are performed in parallel with steps i) and ii).

Amplification of the cells obtained on conclusion of step iii) is performed by seeding said cells in 48-well plates at 10 000 and 15 000 cells/well.
a step v) of placing a chemical substance (S) or a chemical composition (C) in contact with the cell medium obtained in step iv),
a step vi) of placing the MCR20e conditioned medium obtained in step ii) in contact with the “young” or “R6” cells in the amplification phase obtained in step iii),

In this step d), the MCR20 conditioned medium obtained in step ii) is placed in contact with the “young” or “R6” cells obtained in step v) for 48 hours.
a step vii) of measuring the amount of extracellular interleukin IL-6 present in the culture supernatant and measuring the β-galactosidase activity present in the cells obtained from step vi).
a step viii) of comparing the levels of expression of extracellular IL-6 and of β-galactosidase
   More precisely, the following is calculated in the context of this step viii):
   The ratio R1=[(N10−N1i)×100]/[(N10−N1)] for the extracellular interleukin IL-6 with:
N1 corresponding to the amount of IL-6 measured in the culture medium obtained on conclusion of the process as described above, without performing either step v) or step vi) of the process (cells at the R6 passage, untreated and not combined with the conditioned cells of the R20 passage),
N10 corresponding to the amount of IL-6 measured in the culture medium obtained on conclusion of the process as described above, without performing step v) of the process (cells at the R6 passage, untreated, and combined with the conditioned cells of the R20 passage),
N1i corresponding to the amount of IL-6 measured in the culture medium obtained on conclusion of the process as described above, when a substance or a composition (i) is used in step v) of the process (cells at the R6 passage, untreated and not combined with the conditioned cells of the R20 passage).
   The ratio R2=[(N20−N2i)×100]/[(N20−N2)] for the extracellular interleukin IL-6 with:
N2 corresponding to the amount of β-galactosidase measured in the culture medium obtained on conclusion of the process as described above, without performing either step v) or step vi) of the process (cells at the R6 passage, untreated and not combined with the conditioned cells of the R20 passage),
N20 corresponding to the amount of β-galactosidase measured in the culture medium obtained on conclusion of the process as described above, without performing step v) of the process (cells at the R6 passage, untreated, and combined with the conditioned cells of the R20 passage),
N2i corresponding to the amount of β-galactosidase measured in the culture medium obtained on conclusion of the process as described above, when a substance or a composition (i) is used in step v) of the process (cells at the R6 passage, untreated and not combined with the conditioned cells of the R20 passage).
   B]— Evaluation of Substances and Compositions According to the Process that is the Subject of the Present Invention

Composition (CA1) was prepared by extraction using a predried and screened powder of the red alga Asparagopsis armata, with a 1,3-propanediol/water mixture (40/60 v/v) so as to obtain an extract of Asparagopsis armata at 1.2% in said solvent.

Composition (CA2) was prepared by maceration in ethanol at a temperature of 20° C. for a period of 2 hours. The extract thus obtained is evaporated and taken up in methanol, which causes the minerals present to precipitate, this precipitate being removed by filtration. The filtrate is then evaporated and then purified by semi-preparative HPLC (Phenomenex™ C18 column) on an H₂O/CH₃CN solvent gradient, and the collected fraction was saved, and then analyzed to determine an amount of mycosporin-like amino acid in a proportion of 50 mg/L of palythine equivalent.

The results obtained for each of the compositions, following the implementation of the process described previously, are collated in Table 2 below:

Compositions (CA1) and (CA2) were then diluted in 1,3-propanediol to a proportion of 0.1% by mass.

TABLE 2

| | | β-galactosidase |
|---|---|---|
| | | (number of “blue” |
| | Extracellular IL-6 | positive cells/total |
| Products tested | (pg/μg of proteins) | number of cells) |
| Untreated R6 passage “young” cells, not placed in | N1 = 8.96 ± 0.37 | N2 = 3.04 ± 0.78 |
| contact with the MCR20 conditioned medium | | |
| produced by the R20 passage “senescent” cells | | |
| Untreated R6 passage “young” cells, after placing in | N1₀ = 41.38 ± 9.11 | N2₀ = 9.66 ± 1.65 |
| contact with the MCR20 conditioned medium | | |
| produced by the R20 passage “senescent” cells | | |
| R6 passage “young” cells, after treatment with vitamin | N1i = 27.31 ± 3.87 | n.r. |
| C and placing in contact with the MCR20 conditioned | R1 = 43.4 | |
| medium produced by the R20 passage “senescent” | | |
| cells | | |
| R6 passage “young” cells, after treatment with vitamin | n.r. | N2i = 2.99 ± 0.45 |
| E(*) and placing in contact with the MCR20 | | R2 = 100.75 |
| conditioned medium produced by the R20 passage | | |
| “senescent” cells | | |
| R6 passage “young” cells, after treatment with | N1i = 29.81 ± 3.30 | N2i = 3.20 ± 0.61 |
| Composition (CA1) and placing in contact with the | R1 = 35.7 | R2 = 97.6 |
| MCR20 conditioned medium produced by the R20 | | |
| passage “senescent” cells | | |
| R6 passage “young” cells, after treatment with | N1i = 29.45 ± 0.25 | N2i = 4.41 ± 1.54 |
| Composition (CA2) and placing in contact with the | R1 = 36.8 | R2 = 79.3 |
| MCR20 conditioned medium produced by the R20 | | |
| passage “senescent” cells | | |
| R6 passage “young” cells, after treatment with 1,3- | N1i = 37.05 ± 2.57 | N2i = 13.40 ± 2.69 |
| propanediol and placing in contact with the MCR20 | R1 = 13.35 | R2 = −56.5 |
| conditioned medium produced by the R20 passage | | |
| “senescent” cells | | |

(*)Vitamin E is D-α-tocopheryl acetate

### Analysis of the Results

The ratios R1 are greater than 30 both for vitamin C used as reference compound and for compositions (CA1) and (CA2) according to the invention.
The ratios R1 are greater than 70 both for vitamin E used as reference compound and for compositions (CA1) and (CA2) according to the invention.

These compositions (CA1) and (CA2) may be selected for protecting against contagious aging or the bystander effect.

### BIBLIOGRAPHY

[1]: Campisi, J. and d'Adda di Fagagna F., “Cellular senescence: when bad things happen to good cells.” Nat. Rev., Mol. Cell Biol., 2007, 8, 729-740
[2]: Coppe J. P. et al., “Senescence-associated secretory phenotypes reveal cell-nonautonomous functions of oncogenic RAS and the p53 tumor suppressor”, PloS Biol., 2008, 6, 2853-2868
[3]: Passos J. F. et al., “A feedback between p21 and reactive oxygen production is necessary for cell senescence”, Mol. Syst. Biol, 2010, 6, 347
[4]: Nelson G., Wordsworth J., Wang C., Jurk D., Lawless C., Martin-Ruiz C. and Von Zglinicki T.: “A senescent cell bystander effect: senescence-induced Senescence”. Aging Cell, 2012, 11, 345-349.
[5]: Redmond R. W., Rajadurai A., Udayakumar D., Sviderskaya E. V., Tsao H. “Melanocytes are selectively vulnerable to UVA-mediated Bystander Oxidative Signaling.” J. Invest. Dermatol. 2014, 134, pp. 1083-1090.

1. A process for evaluating the capacity of a chemical substance (S) or of a chemical composition (C) for preventing or slowing down the appearance of the signs of aging of human skin or the lips or for eliminating said signs; said process comprising:
a step a) of placing the chemical substance or the chemical composition in contact with “young” human dermal fibroblast cells taken from a culture medium at the R6 passage,
a step b) of placing human senescent dermal fibroblast cells in contact with “young” fibroblast cells obtained from step a), and
a step c) of measuring the entry into senescence of the “young” fibroblast cells and of comparison with a reference value.

2. The process as claimed in claim 1, wherein the senescent cells are human dermal fibroblast cells taken from a culture medium at the R20 passage.

3. The process as claimed in claim 1, wherein the senescent cells are human dermal fibroblast cells which have been taken from a culture medium at the R19 passage before undergoing amplification.

4. The process as claimed in claim 1, wherein the “young” human dermal fibroblast cells taken from a culture medium at the R6 passage are obtained from an amplification of “young” human dermal fibroblast cells taken from a culture medium at the R5 passage.

5. The process as claimed in claim 1, wherein step c) of measuring the entry into senescence of the “young” fibroblast cells comprises the use of at least two biological markers:
at least one biological marker (M1) for SASP/SMS, and
at least one biological marker (M2) for senescence.

6. The process as claimed in claim 5, wherein the biological marker (M1) for SASP/SMS is chosen from the elements of the group consisting of proinflammatory cytokines, extracellular interleukin-1 (IL-1), extracellular interleukin-6 (IL-6), extracellular interleukin-8 (IL-8), growth factors, matrix degradation enzymes, and reactive oxygen species or ROS.

7. The process as claimed in claim 5, wherein the biological marker (M2) for senescence is chosen from the elements of the group consisting of β-galactosidase, persistent DNA damage, and apoptosis resistance.

8. The process as claimed in claim 5, wherein the biological marker (M1) is interleukin-6 and the biological marker (M2) is the β-galactosidase enzyme.

9. The process as claimed in claim 5, wherein, in step c), the levels of expression of the two biological markers (M1) and (M2) are compared with at least one reference level of expression for each of these two biological markers.

10. The process as claimed in claim 1, wherein the signs of aging of human skin or the lips are chosen from
the appearance of wrinkles, fine lines or impairment of the microrelief; and/or
the appearance of lack of elasticity and/or tonicity; and/or
the appearance of lack of density and/or firmness.

11. A cosmetic composition (CA) comprising, per 100% of mass, from 0.1% to 25% by mass of at least one compound of general formula (I): 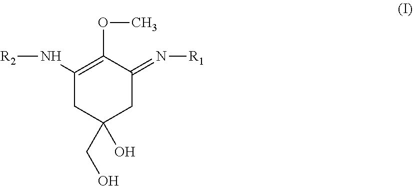
in which:
R₁ represents the radical of formula (Ia)
—CH₂—CO₂H (Ia),
and R₂ represents a hydrogen atom, or
R₁ represents the radical of formula (Ib)
—CH(CO₂H)—CH₂—CH₂—OH (Ib),
and R₂ represents a hydrogen atom, or
R₁ represents the radical of formula (Ia) and R₂ represents the hydroxyethyl radical (Ic):
HO—CH₂—CH₂— (Ic), or
R₁ represents the radical of formula (Ia) and R₂ represents the hydroxymethylethyl radical (Id):
HO—C(CH₃)(CH₂—CH₃) (Id), or
R₁ represents the radical of formula (Ia) and R₂ represents the hydroxypropyl radical (Ie):
HO—CH₂—CH₂—CH₂— (Ie), or
R₁ represents the radical of formula (Ia) and R₂ represents the (E)-propenyl radical (If), or
R₁ represents the radical of formula (Ia) and R₂ represents the (Z)-propenyl radical (Ig), or
R₁ and R₂ both represent the radical of formula (Ia), or
R₁ represents the radical of formula (Ii)
—CH(CO₂H)—CH(OH)—CH₃ (Ii)
and R₂ represents a hydrogen atom, or
R₁ represents the radical of formula (Ii) and R₂ represents a methyl radical, or
R₁ represents the radical of formula (Ib) and R₂ represents a methyl radical, or
R₁ represents the radical of formula (Ia) and R₂ represents the radical of formula (Ij)
—CH(CO₂H)—CH₃ (Ij), or
R₁ represents the radical of formula (Ia) and R₂ represents the radical of formula (Ib), or
R₁ represents the radical of formula (Ia) and R₂ represents the radical of formula (Ik)
—CH(CO₂H)—CH(CH₃)₂ (Ik), or
R₁ represents the radical of formula (Ia) and R₂ represents the radical of formula (Ii), or
R₁ represents the radical of formula (Ia) and R₂ represents the radical of formula (Il)
—CH(CO₂H)—CH₂(CO₂H) (Il), or
R₁ represents the radical of formula (Ia) and R₂ represents the radical of formula (Im)
—CH₂—CH═CH—CO₂H (Im).

12. A method for preventing or slowing down the appearance of the signs of aging of human skin or the lips or for eliminating said signs, comprising applying an effective amount of the cosmetic composition of claim 11.

13. A cosmetic formulation suitable for topical use comprising at least one cosmetically acceptable excipient and an effective amount of the cosmetic composition (CA) as defined in claim 11.

14. A method for preventing or slowing down the appearance of the signs of aging of human skin or the lips or for eliminating said signs, comprising applying an effective amount of the cosmetic formulation of claim 13.

15. The process as claimed in claim 2, wherein the “young” human dermal fibroblast cells taken from a culture medium at the R6 passage are obtained from an amplification of “young” human dermal fibroblast cells taken from a culture medium at the R5 passage.

16. The process as claimed in claim 3, wherein the “young” human dermal fibroblast cells taken from a culture medium at the R6 passage are obtained from an amplification of “young” human dermal fibroblast cells taken from a culture medium at the R5 passage.

17. The process as claimed in claim 2, wherein step c) of measuring the entry into senescence of the “young” fibroblast cells comprises the use of at least two biological markers:
at least one biological marker (M1) for SASP/SMS, and
at least one biological marker (M2) for senescence.

18. The process as claimed in claim 3, wherein step c) of measuring the entry into senescence of the “young” fibroblast cells comprises the use of at least two biological markers:
at least one biological marker (M1) for SASP/SMS, and
at least one biological marker (M2) for senescence.

19. The process as claimed in claim 4, wherein step c) of measuring the entry into senescence of the “young” fibroblast cells comprises the use of at least two biological markers:
at least one biological marker (M1) for SASP/SMS, and
at least one biological marker (M2) for senescence.

20. The process as claimed in claim 6, wherein the biological marker (M2) for senescence is chosen from the elements of the group consisting of β-galactosidase, persistent DNA damage, and apoptosis resistance.
